(19) United States
(12) Patent Application Publication
(10) Pub. No.: US 2016/0279057 A1
(43) Pub. Date: Sep. 29, 2016

(54) Azelastine Formulations For Intranasal Nebulization And Irrigation

(71) Applicant: CMPD LICENSING, LLC, Conroe, TX (US)

(72) Inventor: Jay Richard Ray, II, Conroe, TX (US)

(21) Appl. No.: 14/671,511

(22) Filed: Mar. 27, 2015

(51) Int. Cl. A61K 9/00; A61K 45/06; A61K 31/55

(52) U.S. Cl. CPC A61K 9/0078; A61K 31/55; A61K 45/06

(57) ABSTRACT

A method of treating allergic rhinitis includes nebulizing a formulation including azelastine in an aqueous solution. Nebulizing the formulation produces nebulized particles for intranasal delivery to the patient. Nebulizing may be performed using a small or large particle nebulizer.

### FIELD

The present disclosure relates to intranasal formulations for treating rhinologic conditions, in particular, the present disclosure relates to nebulizer and irrigation formulations that include azelastine for treatment of rhinologic conditions.

### BACKGROUND

Respiratory tract conditions are extremely common ailments of the human experience and include rhinologic conditions, infections, and other obstructions to respiration. One reason for this frequency is constant exposure of respiratory surfaces to the external environment. For example, foreign matter such as debris, microorganisms, viruses, biological matter, and even harsh environmental conditions may enter the body during respiration, irritating or infecting respiratory tract surfaces or even the internal body. Overtime, the human body has also evolved defenses designed to protect the body from this exposure. These defenses include mucous lining and immune responses such as inflammation and increased mucous production or viscosity. Diseases and abnormalities such as chronic obstructive pulmonary disease (COPD), asthma, rhinitis, and various allergies may also include undesirable triggering or modulation of such defenses, e.g., excessive immune responses that cause bronchioconstriction or excessive mucous production or thickening.

Often times an initial treatment objective of respiratory tract conditions is to relieve the obstruction and restore unobstructed respiration by increasing drainage or relieving inflammation. Further objectives may be directed to relieving discomfort or treating the underlying condition. Treatments may include localized application or action of medications, e.g., using nasal spray or metered inhaler. However, respiratory tract conditions may manifest at multiple locations complicating targeted delivery of medication where needed, thus, systemic delivery routes such as oral or intravenous administration, have also been used. Causes of respiratory tract conditions are also numerous and identification of a precise cause may be difficult, especially when multiple conditions are present.

What is needed are treatment solutions for respiratory tract conditions designed to improve treatment outcomes.

### SUMMARY

In one aspect, a method of treating a subject for allergic rhinitis comprises nebulizing a nebulizer formulation comprising a pharmaceutically effective amount of azelastine or a pharmaceutically equivalent salt or derivative thereof in an aqueous solution. The nebulizing produces nebulized particles of the nebulizer formulation for intranasal delivery to the subject. In one embodiment, the nebulizing comprises using a small particle nebulizer to produce nebulized particles. In another embodiment, the nebulizing comprises using a large particle nebulizer to produce large nebulized particles.

In one embodiment, the nebulizer formulation further comprises at least one of an anti-infective agent, a corticosteroid, a leukotriene receptor antagonist, and a mast cell stabilizer in the aqueous medium. The anti-infective agent may comprise one or both of an anti-microbial and an anti-fungal. The anti-microbial may comprises at least one of a quinolone, a sulfa-based anti-microbial, mupirocin, and vancomycin. The anti-fungal may comprise at least one of an azole and amphotericin B. The corticosteroid may comprise at least one of fluticasone, budesonide, mometasone, and betamethasone. For example, the corticosteroid may comprise about 3 mg fluticasone. The corticosteroid may comprise about 0.25 mg or about 0.5 mg budesonide. The mast cell stabilizer may comprise cromolyn sodium.

In another aspect, a nebulizer formulation for treating allergic rhinitis includes a nebulizer formulation comprising a pharmaceutically effective amount of azelastine or a pharmaceutically equivalent salt or derivative thereof in an aqueous solution, and at least one of an anti-infective agent, leukotriene receptor antagonist, and a mast cell stabilizer.

In one embodiment, the anti-infective agent comprises one or both of an anti-microbial and an anti-fungal. In a further embodiment, the anti-microbial may comprise at least one of a quinolone, a sulfa-based anti-microbial, mupirocin, and vancomycin. The anti-fungal may comprise at least one of an azole and amphotericin B. The mast cell stabilizer may comprise cromolyn sodium. The nebulizer formulation may further comprise a corticosteroid, wherein the corticosteroid comprises at least one of fluticasone, budesonide, mometasone, and betamethasone. For example, the corticosteroid may comprise about 3 mg fluticasone. The corticosteroid may also comprise about 0.25 mg or about 0.5 mg budesonide.

In yet another aspect, a method of treating a subject for allergic rhinitis comprises applying an irrigation formulation to the nasal cavity of the subject to intranasally deliver therein a pharmaceutically effective amount of azelastine or a pharmaceutically equivalent salt or derivative thereof and at least one additional active agent. The azelastine and at least one additional active agent may be applied within an aqueous liquid medium. At least one additional active agent may comprise one or more of an anti-infective agent, a corticosteroid, leukotriene receptor antagonist, and a mast cell stabilizer. The anti-infective agent may comprise one or both of an anti-microbial and an anti-fungal. In one embodiment, the anti-infective agent comprises at least one of a quinolone, a sulfa-based anti-microbial, mupirocin, vancomycin, an azole, and amphotericin B. The corticosteroid may comprise at least one of fluticasone, budesonide, mometasone, and betamethasone. For example, the corticosteroid may comprise about 3 mg fluticasone, about 0.25 mg of budesonide, or about 0.5 mg budesonide. The mast cell stabilizer may comprise cromolyn sodium.

### DESCRIPTION

In various embodiments, the present disclosure is directed to methods and pharmaceutical compositions for treatment of conditions of the respiratory tract. Conditions may include one or more ailments or symptomologies affecting the respiratory tract. The methods of treatment and compositions may generally comprise a pharmaceutically effective amount of azelastine, which those having skill in the art will appreciate may include salts, pharmaceutical equivalents, or derivatives of azelastine. For brevity, however, such salts, equivalents, and derivatives may be referred to herein as azelastine. For example, the composition may comprise a pharmaceutically effective amount of azelastine, which may be provided as an equivalent pharmaceutically effective amount of azelastine hydrochloride.

Azelastine or azelastine hydrochloride is an antihistamine formulated as a metered spray solution (0.137 mL mean volume containing 137 mcg of azelastine hydrochloride (equivalent to 125 mcg of azelastine base)) for intranasal administration. Azelastine nasal spray contains 0.1% azelastine hydrochloride in an aqueous solution at pH 6.8±0.3. It also contains benzalkonium chloride (125 mcg/mL), edetate disodium, hypromellose, citric acid, dibasic sodium phosphate, sodium chloride, and purified water. Azelastine hydrochloride occurs as a white crystalline powder with a bitter taste that is sparingly soluble in water, methanol, and propylene glycol and slightly soluble in ethanol, octanol, and glycerin.

The composition may generally comprise azelastine alone or in combination with one or more additional pharmaceutical components provided, e.g., dispensed, administered, or delivered, within a treatment solution. In one embodiment, however, one or more pharmaceutical components may be provided to a user, e.g., a medical professional or patient, as a dry powder for addition to a base such as an aqueous liquid to form a treatment solution prior to administration.

In some embodiments, azelastine may be provided within a treatment solution. One or more additional pharmaceutical components such as additional active agents may be added to the treatment solution prior to dispensing or administration. For example, the treatment solution may be in the form of a mixture, suspension, emulsion, or solution wherein the one or more pharmaceutical components are dissolved, mixed, suspended, or otherwise dispersed.

The present disclosure is also directed to methods of delivering the composition. According to various embodiments, the composition may comprise an irrigation or nebulizer formulation configured for intranasal administration. Such formulations may be configured, for example, for delivery to target sites for treatment by irrigation or nebulization. For example, the composition, when prepared for administration, may be formulated in a dose form comprising a treatment solution suitable for administration to the nasal cavity. In one embodiment, the treatment solution is configured to be delivered by irrigation at the nasal cavity. In another embodiment, the treatment solution is configured to be delivered by a nebulizer to produce aerosol particles or droplets suitable for inhalation and targeted deposition of such aerosol along the respiratory tract. In some embodiments, the treatment solution may be nebulized using a nebulizer configured to produce small or large aerosol particles, with respect to the particle size dispersion generated by the nebulization, e.g., using a Nasoneb, Sinustar, or other suitable nebulizer. Various embodiments may further comprise a base fluid, carrier, which may include delivery vehicles, excipients, or additional active agents.

The composition may comprise azelastine alone or in concert with other active pharmaceutical components. For example, the composition may be a compounded pharmaceutical formulation comprising azelastine and one or more additional active agents. Additional active agents, for example, may be included in the composition to treat a same or different rhinologic condition as the azelastine component. For example, other active agents may be included to treat related or secondary conditions, potential side effects or adverse reactions of the treatment, or other condition accompanying the rhinologic condition initially treated.

In various embodiments, additional active agents may comprise one or more of the following or their equivalents, derivatives, salts, or analogs, including, an anti-infective or antimicrobial agent (e.g., antibiotic, antiviral, antiparacytic, antifungal, etc.), corticosteroid, anti-inflammatory, decongestant (e.g., vasoconstrictor), mast cell stabilizer, leukotriene receptor antagonist, cycloplegic or the like, or another suitable drug such as an agent for blocking or modulating immune system or allergic responses (e.g., antihistamine, cytokine inhibitor, leukotriene inhibitor, IgE inhibitor, immunomodulator).

Various embodiments of the composition may comprise azelastine and one or more active agents comprising one or more anti-infective agents. Exemplary anti-infective agents may include, but are not limited to, antimicrobials and antifungals. Some embodiments may include an antimicrobial comprising one or more antibiotics selected from quinolones or sulfa-based agents. These or other embodiments may include an antimicrobial comprising one or more antibiotics selected from mupirocin, vancomycin, tetracycline, chlortetracycline, bacitracin, neomycin, polymyxin, gramicidin, cephalexin, oxytetracycline, chloramphenicol, rifampicin, ciprofloxacin, tobramycin, gentamycin, erythromycin, penicillin, sulfonamides, sulfadiazine, sulfacetamide, sulfamethizole, sulfisoxazole, nitrofurazone, sodium propionate, or other antibiotics or derivatives thereof. Some embodiments may include an antimicrobial comprising one or more antifungals selected from amphotericin B or nystatin. These or other embodiments may include an antimicrobial comprising one or more antifungals comprising an azole. Azoles may include azoles known to those skilled in the art such as clotrimazole, econazole, oxiconazole, ketoconazole, miconazole, sulconazole, fluconazole, itraconazole, voriconazole. Antimicrobials may also include other antifungals such as terbinafine, flucytosine, ciclopirox, or other suitable antifungal or derivatives thereof.

Various embodiments of the composition may comprise azelastine and one or more active agents comprising one or more corticosteroids. Exemplary corticosteroids may include but are not limited to fluticasone, budesonide, mometasone, betamethasone, triamcinolone, dexamethasone, fluocinolone, cortisone, prednisolone, or other suitable corticosteroid or derivatives thereof.

Various embodiments of the composition may also comprises azelastine and one or more active agents comprising one or more anti-inflammatory agents. Anti-inflammatory agents may include hydrocortisone, hydrocortisone acetate, dexamethasone 21-phosphate, fluocinolone, medrysone, methylprednisolone, prednisolone 21-phosphate, prednisolone acetate, fluoromethalone, betamethasone, triamcinolone, triamcinolone acetonide, and non-steroidal anti-inflammatories (NSAIDs) such as salicylate, indomethacin, ibuprofen, diclofenac, flurbiprofen, piroxicam indomethacin, ibuprofen, naxopren, piroxicam and nabumetone.

Various embodiments of the composition may comprises azelastine and one or more active agents comprising one or more leukotriene receptor antagonists, for example, montelukast, zafirlukast, pranlukast, zileuton, or pharmaceutically acceptable derivatives thereof.

When the composition comprises azelastine and one or more additional antihistamines, the dose of one or more antihistamines to be delivered may be reduced by at least 5%, at least 10%, at least 15%, at least 20%, at least 25%, at least 30%, at least 40%, at least 50%, at least 60%, at least 70%, at least 80%, at least 90%, or more for conventional dose regimens of the antihistamine alone. The actual reduction in dose will depend upon the nature and amount of the azelastine, other antihistamines, or similarly acting active agents being administered, the symptom relief desired, and other factors set forth elsewhere in this application that are typically considered in treating a disease or condition. The amount of the compounded formulation administered according to this method may also depend upon the factors set forth above, as well as the nature and amount of the one or more additional antihistamines being administered in conjunction with the azelastine component of the compounded formulation. In certain embodiments, the amount of azelastine administered in this method is less than 5%, less than 10%, less than 15%, less than 20%, less than 25%, less than 30%, less than 40%, less than 50%, less than 60%, less than 70%, less than 80%, or less than 90% of the dose of the compounded formulation required to produce symptom relief without conjoint administration with an additional antihistamine.

The composition may include an aqueous treatment solution comprising a base liquid base solution or solvent. The treatment solution may include a solution, mixture, emulsion, or suspension, for example, wherein the active agents mixed, dissolved, suspended, dispersed, or otherwise within the base liquid.

The compositions may comprise one or more of the listed active agents described herein and may further include one or more pharmaceutically acceptable excipients. In other embodiments, however, the formulations consist of the one or more of the listed ingredients and one or more pharmaceutically acceptable excipients. Those skilled in the art will appreciate that the choice of excipients may depend, at least in part, on the active agents selected, condition treated, manner of administration, as well as patient preferences. Exemplary excipients may be configured to assist in the release, dispersion, solubility, or the delivery of one or more of the active agents or to modify taste. For example, excipients may include one or more of diluents, dispersants, preservatives, solvents, co-solvents, wetting agents, buffering agents, humectants, permeation enhancer, emollient, sweetening agents, anti-foaming agents, thickening agents, or flavoring agents, for example.

The treatment solution may comprise a topical preparation formulated for application to an external or internal body surface such skin or mucosal surfaces of the respiratory tract. The topical preparations may be formulated to act at the tissue surface or absorb for local action. In some embodiments, however, the topical preparations may include an aspect of systemic action.

It is to be appreciated that the compositions described herein which employ a base for use in pharmaceutical compounding and manufacturing of topical preparations, may be used for the preparation of compositions for topical administration for appropriate conditions, for example, in some embodiments, the pharmaceutically acceptable excipients or additives include a base for use in pharmaceutical compounding and manufacturing of topical preparations. Such base compositions are known to those skilled in the art. In a preferred embodiment, the base is the PCCA base having the name LoxaSperse. LoxaSperse is an excipient base powder manufactured by PCCA (Houston, Tex.) used as a chemical dispersing or solubilizing agent in irrigation or nebulization formulations, improving the solubility and dispersibility of poorly water soluble Active Pharmaceutical Ingredients (APIs) or agents. LoxaSperse is a blend of specially micronized xylitol with an optimized ratio of micronized poloxamers, designed to improve the dispersibility and solubility of APIs.

In various embodiments, the composition comprises a treatment solution comprising azelastine (azelastine hydrochloride) in a powder form. The powdered form may be dispensed for later addition to a liquid to formulate an aqueous or non-aqueous solution or suspension suitable for the desired method of delivery. The powdered form may include one or more additional active agents or excipients. The liquid may similarly include one or more additional active agents or excipients. In some embodiments, the treatment solution may comprise azelastine hydrochloride nasal spray alone or in combination with one or more additional active agents or excipients. For example, in one embodiment, additional active agents or excipients as described above may be combined to azelastine hydrochloride nasal spray to formulate the treatment solution.

In various embodiments, the composition may comprise a treatments solution dispensed as a liquid preparation or dosage form suitable for intranasal administration via irrigation or nebulization. The liquid preparation may be in ready-use format requiring only that the formulation be transferred to an administration or delivery device, such as a nebulizer, which may include small or large particle nebulizer, or an irrigator, which may be a pressure assisted irrigator.

In other embodiments, the composition may be dispensed as a ready-to-mix powder composition comprising azelastine. The ready-to-mix powder composition may also include one or more additional active agents or excipients. The patient may be directed to mix the ready-to-mix powder composition in a base liquid prior to administration. In various embodiments, the identity or volume of the base liquid may define the suitability of the resulting solution for the desired method of delivery for intranasal administration, e.g., nebulization or irrigation. In some instances, one or more excipients, such as flavoring, wetting agents, solubility enhancers, or dispersants, for example, may be included in the base liquid or ready-to-mix powder.

The composition may be administered to a patient to treat conditions of the respiratory tract. Conditions may include one or more ailments or symptomologies of the respiratory tract. Treatments may be directed to, for example, ailments or symptomologies arising from allergens, infections, or diseases affecting the respiratory tract or its functions. For example, the compositions may be used to treat infections of the lower or upper respiratory tract. The respiratory tract infection treated may include a condition caused by or associated with a bacterial, viral, or fungal infection, including a pseudomonas infection or a MRSA infection, influenza and the common cold. Treatments may also be directed to other conditions such as cystic fibrosis, bronchiolitis, bronchiectasis, tracheobronchitis, pneumonia (including ventilator-associated pneumonia, pneumonitis, dyspnea, cough, (recurrent) wheezing, asthma, nasal polyopsis, rhinitis, hormonal rhinitis, allergic rhinitis, upper respiratory infections (Common cold), pulmonary sarcoidosis, anosmia, olfactory (smell) loss, sinus ostia stenosis, aspergilliosis, pulmonary invasive fungal infections, sinusitis, chronic rhinosinusitis, nosocomial lung infections.

In various embodiments, the treatment method includes administering the treatment solution intranasally via a nebulizer. The treatment may be directed to treatment of allergic rhinitis, an infection of the lower respiratory tract, or both. The treatment may also be directed to treatment of allergic rhinitis and one or more of COPD, asthma, hormonal rhinitis, or another breathing obstruction.

As introduced above, the nebulizer solution or dosage form may comprise an aqueous nebulizer solutions for delivery by a nebulizer device, such an intranasal nebulizer. Delivery of the composition using a nebulizer may enhance delivery efficiency and treatment options by providing flexibility with respect to personalization or customization of treatment. Nebulization of the formulation may be by any suitable commercially available nebulizer device.

In some applications, the use of a nebulizer to deliver the composition by inhalation, which in some embodiments comprises intranasal inhalation, may require only simple tidal breathing to receive the designed dose of the active agents. In one embodiment, intranasal administration via nebulization may efficiently deliver the nebulized formulation to the respiratory tract, e.g., the nasal and paranasal sinus cavities. Once delivered, deposits of the formulation or its active agents, which may be in a concentrated state, may form on surfaces of the respiratory tract and apply therapeutic action thereto. Nebulizer delivery systems may beneficially avoid side effects associated with cold medicated irrigations or aerosol-generating powered nasal irrigators.

In one embodiment, the nebulizer solution is formulated for delivery via a small particle nebulizer device or delivery system. The small particle nebulization delivery system may be configured to nebulize the solution to produce small particles or droplets, e.g., having aerosol characteristics, wherein the particle size of the majority of the particles or droplets formed by the nebulization is less than about 10 microns, about 8 microns, about 5 microns, or about 3 microns. For example, in some embodiments, about 60%, 70%, 80%, 90% or greater of the particles or droplets formed by the nebulization are less than about 5 microns. In these or other embodiments, the particles may be produced within a particle size dispersion wherein at least 50%, 60%, 70%, 80%, 90%, or 95% of the particles may be with about 3 microns and about 10 microns, about 3 microns and about 8 microns, about 3 microns and about 5 microns, about 5 microns and about 8 microns, about 5 microns and about 10 microns, or about 8 microns and about 10 microns.

Accordingly, a method of administering the treatment solution comprising a nebulizer solution may comprise using a small particle nebulizer delivery system and nebulizing the formulation to form small particles or droplets. The small particles may then be inhaled into the upper airway and deposit at the paranasal sinus and nasal mucosa. Compared to large particle nebulizer delivery systems, small particle nebulizer delivery systems may be used to deliver a greater fraction of active agents to the pulmonary system. This may increase systemic bioavailability of the active agents. However, when increased systemic bioavailability is not desirable, e.g., when such bioavailability is linked to unwanted side effects, the formulation may be prepared for and delivered by a large particle nebulizer delivery system. While any suitable small particle nebulizer delivery system or device may be used, one suitable device is a PARI or Sinustar intranasal nebulizer.

In one embodiment, the composition comprises a treatment solution configured for delivery via a large particle nebulizer or delivery system. The large particle nebulizer delivery system may include a nebulizer configured to generate particles or droplets wherein the majority of the particles or droplets are larger than about 5 microns, about 10 microns, about 15 microns, about 20 microns or more, such as about 23 microns. In various embodiments, nebulization with a large particle nebulizer produces aerosol particles wherein the majority of particles are greater than about 10 microns, about 15 microns, about 20 microns, or about 25 microns. In these or other embodiments, the particles may be produced within a particle size dispersion wherein at least 50%, 60%, 70%, 80%, 90%, or 95% of the particles may be within about 10 microns and about 25 microns, about 10 microns and about 20 microns, about 10 microns and about 15 microns, about 15 microns and about 25 microns, about 15 microns and about 20 microns, or about 20 microns and about 25 microns.

Accordingly, a method of administering the composition comprising a treatment solution for large particle nebulization may comprises nebulizing the nebulizer solution to form large particles. The large particles may then be inhaled into the nasal and paranasal sinus cavities and for deposition on the frontal recess/sinus, spheno-ethmoid recess, ethmoid cavity, sphenoid and maxillary sinuses, turbinates, middle meatus, and olfactory cleft. The large particle nebulizer delivery system may be configured to provide low volume, high concentration delivery of the formulation.

While any suitable large particle nebulization system or device may typically be used, one suitable device is a NasoNeb® Nasal Nebulizer. Such large particle delivery systems may be employed to deliver a deep, penetrating aerosol to the nasal and paranasal sinus cavities of the patient. Such delivery may include little to no incidental pulmonary delivery of drugs, which may otherwise occur in small particle systems, as described above. For example, in some embodiments, large particle nebulization may provide superior outcomes compared to small particle nebulization to treat the upper respiratory tract, which typically include pulmonary delivery and decreased nasal and paranasal sinus cavity disposition.

In one embodiment, the large particle nebulizer system may be used to nebulize the nebulizer solution to generate large particles for delivery to the respiratory tract via a positive pressure airstream that ensures the components of the composition reach all of the desired nasal and paranasal sinus cavities. The large particle nebulizer system may preferably deliver the large particles such that they are readily filtered by the nose to ensure a large percentage of medication is delivered upon target surfaces where intended and that little or no unintended components of the formulation are delivered to the lungs, thus, reducing the risk of unwanted complications.

In one embodiment, the large particle nebulizer system is configured to deliver a low volume treatment solution comprising the composition to ensure that the active agents of the formulation stay in the nasal cavity. Accordingly, such a system may reduce waste generated by irrigation systems. In one embodiment, the large particle nebulizer system is configured to deliver between 0.2-15 mL of nebulizer solution comprising the unit dose of active agents for retention in the nasal and paranasal sinus cavities. In one embodiment, the large particle nebulization system may also reduce complications associated with repeated exposure to cold fluid irrigation such as exostoses of the paranasal sinus cavities by warming the solution to near room temperature upon nebulization, which may help to avoid the iatrogenic complication of exostoses from cold fluid irrigation.

Administering the nebulizer treatment solution via a large particle nebulizer system may also avoid undesirable complications that may be linked to long term use of small particle nebulization systems, which may include vocal irritation/alterations, chronic cough, antimicrobial resistance, eosinophilic pneumonia, and reduced lung function.

According to one embodiment, the nebulizer formulation is configured for treatment of allergic rhinitis or other rhinologic conditions. In further embodiments, the nebulizer formulation is configured for additional treatments such as infections that may be hidden by thickening mucous, e.g., bacterial or fungal, or even in hormonally regulated rhinitis, e.g., if allergic or other component is present.

According to various embodiments, delivery of the formulation via a small particle size delivery system provides penetration of the formulation or its active agents into the lower respiratory tract. Thus, in contrast to delivery of commercial azelastine hydrochloride nasal spray, the nebulizer formulation may be delivered to target sites of the lower respiratory tract via a small particle nebulizer at which therapeutic action of the active agents may result in thinning of the mucous within the lungs of the patient. Such action may be particularly beneficial when addressing COPD, asthma, or other breathing situations.

The treatment solution may also be configured for administration via intranasal irrigation. In such embodiments, the typical mode of administration may be in flush form or liquid stream form. An example of suitable sinus rinse delivery mechanisms include the NeilMed® Sinus Rinse Bottle, a medical syringe of about 20 to 60 mL in size, and other squeeze bottle irrigation devices. Typically, the intranasal formulation is administered two or three times a day.

Effectiveness of the composition for treatment of respiratory tract conditions wherein the composition comprises a treatment solution administered via a Nasoneb, Sinustar, or irrigation system may provide advantages over conventional azelastine hydrochloride nasal spray compositions. For example, administration of the composition via intranasal nebulization, e.g., using a Nasoneb intranasal nebulizer, or with irrigation may provide the ability to increase positive pressure associated with such irrigation or nebulization. Also, if delivered via a large particle nebulization system including a large particle nebulizer device, the composition thereof will reach the paranasal sinus area in lieu of the frontal area where a nasal spray would reach thereby providing additional and enhanced benefits.

The composition as described herein may further include the ability to deliver azelastine alone or in concert with other medications such as anti-infective (antibiotic, antifungal), corticosteroid, mast cell stabilizer, or other drugs, as described above. For example, in various embodiments, the composition includes one or more anti-infective agents. Preferred anti-infective agents may include anti-microbials such as quinolones, sulfa-based agents, mupirocin, or vancomycin. Preferred anti-infective agents may also include antifungals such as azole or amphotericin B. In various embodiments, the composition includes one or more corticosteroids such as but not limited to fluticasone, budesonide, mometasone, and betamethasone. In one embodiment, the composition may comprise 3 mg fluticasone per unit dose. In another embodiment, the composition may comprise budesonide derived from 0.25 mg or 0.5 mg vials. In various embodiments, the composition includes a mast cell stabilizer such as cromolyn sodium. In one embodiment, the composition comprises one or more of the above agents added to a commercially available supply of azelastine hydrochloride nasal spray, such as to 2 mL.

According to various embodiments described herein, daily dosing, either once daily or divided doses, may include daily doses of about 137 mcg, about 500 mcg, about 1,000 mcg, or more, e.g., up to about 15,000 mcg.

All numerical quantities stated herein are approximate unless stated otherwise, meaning that the term “about” or “approximately” may be inferred when not expressly stated. The numerical quantities disclosed herein may be nominal numerical quantities and are to be understood as not being strictly limited to the exact numerical values recited. Instead, unless stated otherwise, each numerical value is intended to mean both the recited value and a functionally, for example pharmaceutically, equivalent range surrounding that value. All numerical ranges stated herein include all sub-ranges subsumed therein. For example, a range of approximately or about 1 to 10 is intended to include all sub-ranges between and including the recited minimum value of 1 and the recited maximum value of 10. Any maximum numerical limitation recited herein is intended to include all lower numerical limitations. Any minimum numerical limitation recited herein is intended to include all higher numerical limitations. Additionally, in some illustrative embodiments, quantities or ranges may be given. It is to be understood that any such quantity or range is provided as an illustrative example or instance of an embodiment and is not intended to limit that or other embodiments.

This disclosure describes various elements, features, aspects, and advantages of various embodiments of the stopping systems, apparatuses, and methods thereof. It is to be understood that certain descriptions of the various embodiments have been simplified to illustrate only those elements, features and aspects that are relevant to a more clear understanding of the disclosed embodiments, while eliminating, for purposes of brevity or clarity, other elements, features and aspects. Any references to “various embodiments,” “certain embodiments,” “some embodiments,” “one embodiment,” or “an embodiment” generally means that a particular element, feature and/or aspect described in the embodiment is included in at least one embodiment. The phrases “in various embodiments,” “in certain embodiments,” “in some embodiments,” “in one embodiment,” or “in an embodiment” may not refer to the same embodiment.” Furthermore, the phrases “in one such embodiment” or “in certain such embodiments,” while generally referring to and elaborating upon a preceding embodiment, is not intended to suggest that the elements, features, and aspects of the embodiment introduced by the phrase are limited to the preceding embodiment; rather, the phrase is provided to assist the reader in understanding the various elements, features, and aspects disclosed herein and it is to be understood that those having ordinary skill in the art will recognize that such elements, features, and aspects presented in the introduced embodiment may be applied in combination with other various combinations and sub-combinations of the elements, features, and aspects presented in the disclosed embodiments. The present disclosure is not intended to be limited by the percent composition of the examples unless claimed otherwise. Percent compositions are to be understood as being by weight unless specified otherwise.

The phrase “consisting essentially of” limits a composition to the specified materials or steps and those additional, undefined components that do not materially affect the basic and novel characteristic(s) of the composition, such as, for example, additional active ingredients. In still other embodiments, the formulations consist of the listed active ingredients and a pharmaceutically acceptable carrier. “Consisting of” refers to the inclusion of exactly one element of a number or list of elements. The grammatical articles “one”, “a”, “an”, and “the”, as used in this specification, are intended to include “at least one” or “one or more”, unless otherwise indicated. Thus, the articles are used in this specification to refer to one or more than one (i.e., to “at least one”) of the grammatical objects of the article. By way of example, “a component” means one or more components, and thus, possibly, more than one component is contemplated and may be employed or used in an implementation of the described embodiments. Further, the use of a singular noun includes the plural, and the use of a plural noun includes the singular, unless the context of the usage requires otherwise. Additionally, the grammatical conjunctions “and” and “or” are used herein according to their accepted usage.

1. A method of treating a subject for allergic rhinitis, the method comprising:
(i) using a nebulizer to nebulize a composition, wherein the composition comprises an aqueous solution comprising a therapeutically effective amount of azelastine or a pharmaceutically acceptable salt thereof, a corticosteroid, and at least one of an anti-infective agent, a leukotriene receptor antagonist, and mast cell stabilizer; and
(ii) intranasally administering to a subject in need thereof the nebulized composition.

2. The method of claim 1, wherein nebulizing the composition produces nebulized particles for intranasal administration, wherein the particles are less than about 10 microns.

3. The method of claim 1, wherein nebulizing the composition produces nebulized particles for intranasal administration, wherein the particles are greater than about 10 microns.

4. (canceled)

5. The method of claim 1, wherein the anti-infective agent comprises one or both of an anti-microbial agent and an anti-fungal agent.

6. The method of claim 5, wherein the anti-microbial agent comprises at least one of a quinolone, a sulfa-based anti-microbial, mupirocin, and vancomycin.

7. The method of claim 5, wherein the anti-fungal agent comprises at least one of an azole and amphotericin B.

8. The method of claim 1, wherein the corticosteroid comprises at least one of fluticasone, budesonide, mometasone, and betamethasone.

9. The method of claim 8, wherein the corticosteroid comprises about 3 mg fluticasone.

10. The method of claim 8, wherein the corticosteroid comprises about 0.25 mg budesonide or about 0.5 mg budesonide.

11. The method of claim 1, wherein the mast cell stabilizer comprises cromolyn sodium.

12. A composition for treating allergic rhinitis, comprising:
an aqueous solution comprising a therapeutically effective amount of azelastine or a pharmaceutically acceptable salt thereof;
a corticosteroid; and
at least one of an anti-infective agent, a leukotriene receptor antagonist, and a mast cell stabilizer.

13. The composition of claim 12, wherein the anti-infective agent comprises one or both of an anti-microbial agent and an anti-fungal agent.

14. The composition of claim 13, wherein the anti-microbial agent comprises at least one of a quinolone, a sulfa-based anti-microbial, mupirocin, and vancomycin.

15. The composition of claim 13, wherein the anti-fungal agent comprises at least one of an azole and amphotericin B.

16. The composition of claim 12, wherein the mast cell stabilizer comprises cromolyn sodium.

17. The composition of claim 12, wherein the corticosteroid comprises at least one of fluticasone, budesonide, mometasone, and betamethasone.

18. The composition of claim 17, wherein the corticosteroid comprises about 3 mg fluticasone.

19. The composition of claim 17, wherein the corticosteroid comprises about 0.25 mg budesonide or about 0.5 mg budesonide.

20. (canceled)

21. (canceled)

22. (canceled)

23. (canceled)

24. (canceled)

25. (canceled)

26. The method of claim 1, comprising repeating step (i) and step (ii).

27. The method of claim 1, wherein the nebulized composition comprises about 137 mcg azelastine or a pharmaceutically acceptable salt thereof.

28. The method of claim 1, wherein the nebulized composition comprises about 500 mcg azelastine or a pharmaceutically acceptable salt thereof.

29. The method of claim 1, wherein the nebulized composition comprises about 1000 mcg azelastine or a pharmaceutically acceptable salt thereof.

30. The method of claim 1, wherein the aqueous solution comprises benzalkonium chloride, edetate disodium, hypromellose, citric acid, dibasic sodium phosphate, and sodium chloride.

31. The method of claim 1, comprising preparing the composition of step (i).

32. The composition of claim 12, wherein the aqueous solution comprises benzalkonium chloride, edetate disodium, hypromellose, citric acid, dibasic sodium phosphate, and sodium chloride.
